(19) United States
(12) Patent Application Publication
(10) Pub. No.: US 2025/0326808 A1
(43) Pub. Date: Oct. 23, 2025

(54) TREATMENT OF THE CORNEAL ENDOTHELIUM

(71) Applicant: UCL BUSINESS LTD, London, Greater London (GB)

(72) Inventors: Rintra WONGVISAVAVIT, London (GB); Julie DANIELS, London (GB)

(73) Assignee: UCL BUSINESS LTD, London, Greater London (GB)

(21) Appl. No.: 18/875,552

(22) PCT Filed: Jun. 15, 2023
(86) PCT No.: PCT/EP2023/066095

§ 371 Date: Dec. 16, 2024

(30) Foreign Application Priority Data
Jun. 17, 2022 (GB) 2208950.2

(51) Int. Cl. C07K 14/52; A61K 38/00; C12N 5/079

(52) U.S. Cl. CPC C07K 14/522; C12N 5/0621; A61K 38/00; C12N 2501/21

(57) ABSTRACT

The present invention relates to the treatment of corneal endothelial cells in corneal disease and/or dysfunction. Further, the present invention relates to a method of preparing corneal endothelial cells in vitro.

### FIELD OF THE INVENTION

The present invention relates to the treatment of the corneal endothelium in corneal disease and/or dysfunction. Further, the present invention relates to a method of preparing corneal endothelial cells in vitro.

### BACKGROUND OF THE INVENTION

The cornea is a transparent, avascular tissue on the outermost surface of the eye. The sensory function of the eye depends on the transparency of the cornea, which determines the quality of vision and, ultimately, quality of life. Transparency of the cornea depends on both the outer and inner integrity of the corneal epithelium and corneal endothelium (CE), respectively. Corneal endothelial cells (CECs) facilitate the function of maintaining the transparency of the cornea. Adult CEC density is approximately 2000-3000 cells/mm². The number of CECs declines with age approximately 0.6%/year. As CECs do not have regenerative potential in vivo, it is important that they are maintained. Damage or dysfunction of these cells could lead to partial or total blindness.

Various pathologies cause CEC dysfunction and loss, including viral infection, intraocular surgery and Fuchs' endothelial dystrophy (FED). FED is the most common aetiology of corneal endothelial dysfunction and is considered a primary indication for corneal transplantation. FED is highly prevalent as it affects, for example, up to 4% of those over 40 years old in the USA. It is considered that up to 35% of all corneal transplants performed in the United Kingdom are due to FED. CEC loss after cataract and glaucoma surgery is widely discussed as a common cause of postoperative corneal oedema and vision loss. Rates of CEC loss during cataract surgery are around 8.5%. Meanwhile, rates of CEC loss after glaucoma surgery are from 3.1% to 42.6% based on surgical complexity.

Cornea transplantation is the primary treatment for patients with CEC loss or dysfunction due to corneal endothelial diseases. In the past, FED was treated with penetrating keratoplasty (PK), which is a transplantation procedure that replaces the diseased cornea with a full-thickness donor corneal graft. Although corneal transplantation is the standard treatment for patients with corneal endothelial diseases, the shortage of cornea donors remains a significant problem. The number of patients requiring treatment is much greater than that of donors, and their quality of life deteriorates as they wait for treatment. Hence, several alternative techniques have been proposed and examined in the art, such as tissue substitutes using tissue-engineering methods and CEC culture in vitro. However, the attempt to promote CEC proliferation both in vitro and in vivo has proved to be challenging. The CECs are presumed to lack the ability to proliferate because of cell cycle arrest at the G1 phase.

Therefore, there remains a need to identify suitable growth promoting factors based on understanding CEC biology. It is considered that understanding the fundamental biology in this way will lead to much needed alternative treatments for CEC disease and dysfunction.

### SUMMARY OF THE INVENTION

The present invention is defined in the appended claims.

In accordance with a first aspect, there is provided a CXC chemokine for use in the treatment and/or prevention of damage or dysfunction of the corneal endothelium (CE). There is also provided a method for treating and/or preventing damage or dysfunction of the CE, comprising administering to a subject in need thereof, a therapeutically effective amount of CXC chemokine.

In accordance with a second aspect, there is provided a method of preparing corneal endothelial cells (CECs) in vitro, wherein the method comprises:
a. peeling the Descemet membrane together with the endothelial cells from donor cornea,
b. isolating the primary CECs from the Descemet membrane using one or more cell dissociation reagent(s); and
c. culturing the CECs in medium comprising CXCL5.

Certain embodiments of the present invention may provide one or more of the following advantages:
providing treatment of CE dysfunction/disease without the need for donor corneas, and therefore addressing the problem of limited numbers of donor corneas;
providing effective and non-invasive treatment of CE dysfunction/disease;
providing non-invasive treatment of CE dysfunction/disease;
providing corneal grafts for transplantation; and
addressing the shortage of high-quality donor corneas.

The details, examples and preferences provided in relation to any particular one or more of the stated aspects of the present invention apply equally to all aspects of the present invention. Any combination of the embodiments, examples and preferences described herein in all possible variations thereof is encompassed by the present invention unless otherwise indicated herein, or otherwise clearly contradicted by context.

### BRIEF DESCRIPTION OF THE DRAWINGS

The invention will further be illustrated by reference to the following figures:

FIG. 1 Significantly greater cell proliferation was observed in the group of 20 ng/ml of ENA-78 compared with control at day 3 using the MTT assay. Data is presented as mean±SEM. Two-way ANOVA with post-hoc Tukey HSD test was performed. ** Significant difference (p-value<0.01).

FIG. 2 Significantly greater cell number was observed in the group of 20 ng/mL of ENA-78 compared with control at day 3 using cell counting. Data is presented as mean±SEM. Two-way ANOVA with post-hoc Tukey HSD test was performed. * Significant difference (p-value<0.05).

FIG. 3 10 ng/ml of FGFb was used as a positive control. CEC proliferation in FGFb group increased significantly on day 3 compared with control using the MTT assay. Data is presented as mean±SEM. Two-way ANOVA with post-hoc Tukey HSD test was performed. ** Significant difference (p-value<0.01).

FIG. 4 10 ng/ml of FGFb was used as a positive control. Number of CEC in FGFb group increased significantly on day 3 compared with control using cell counting. Data is presented as mean±SEM. Two-way ANOVA with post-hoc Tukey HSD test was performed. ** Significant difference (p-value<0.01).

FIG. 5 Wound borders were identified (black lines). Wounded area was determined as the area between 2 borders, while wound closure was determined as the difference between the wounded area at 0 hours and 72 hours post injury. The highest wound closure was found in FGFb>ENA-78>negative control at 72 hours post wounding, respectively. Scale bar=50 μm.

FIG. 6 Percent of wound closure of HCECLs treated with ENA-78. Significantly greater percent of wound closure compared with the negative control group was found in the ENA-78 treatment group on day 3 post injury. Data is presented as mean±SEM. Independent sample T-test was performed. * Significant difference (p-value<0.05).

FIG. 7 Percent of wound closure of HCECLs treated with FGFb (positive control). HCECL in FGFb showed a significantly greater percentage of wound closure on day 3 post injury compared with the negative control. Data is presented as mean±SEM. Independent sample T-test was performed. * Significant difference (p-value<0.05).

FIG. 8 Cell proliferation significantly increased in the groups of ENA-78 treatment, and CXCR2 Ab treatment compared with the negative control. Cell proliferation significantly decreased in the group of ENA-78+CXCR2 Ab compared with the negative control. A significantly greater cell proliferation was found in the ENA-78 treatment group compared with the ENA-78+CXCR2 Ab treatment group. Data is presented as mean±SEM. One-way ANOVA with post-hoc Tukey HSD test was performed. * Significant difference (p-value<0.05). ** Significant difference (p-value<0.01).

FIG. 9 Wound borders of HCECLs were identified (black lines). Wounded area was determined as the area between 2 borders, while wound closure was determined as the difference between the wounded area at 0 hours and 72 hours post injury. The highest wound closure was found in ENA-78>CXCR2 Ab>ENA-78+CXCR2>negative control at 72 hours post wounding. Scale bar=50 μm.

FIG. 10 Percent of wound closure was similar at 24 hours post injury in the different treatment groups. ENA-78, ENA-78+CXCR2 Ab and CXCR2 Ab treatment groups showed significantly greater percent of wound closure compared with the negative control at 48 hours post injury. Meanwhile, percent of wound closure in the ENA-78 treatment group was greater than in the ENA-78+CXCR2 Ab and CXCR2 Ab treatment groups at 48 and 72 hours post injury. Data is presented as mean±SEM. One-way ANOVA with post-hoc Tukey HSD test was performed. ** Significant difference (p-value<0.01).

FIG. 11 Western blot was performed to detect p-ERK1/2 protein in HCECL cultured in 20 ng/mL ENA-78 supplemented medium for 0 (control), 30, 60 and 120 minutes. P-ERK1/2 expression levels are presented relative to the GAPDH control. The highest expression of p-ERK1/2 was found at 30 minutes followed by 60- and 120-minutes post treatment, respectively. Data is presented as mean±SEM. One-way ANOVA with post-hoc Tukey HSD test was performed. *Significant difference (p-value<0.05) between HCECL cultured in ENA-78 supplemented medium for 120 minutes and negative control. ** Significant difference (p-value<0.01) between HCECL cultured in ENA-78 supplemented medium for 30 and 60 minutes and control.

FIG. 12 Image of Western blot detecting p-ERK1/2 protein expression in HCECL cultured in 20 ng/mL ENA-78 supplemented medium for 0 (control), 30, 60 and 120 minutes, including a GAPDH control.

FIG. 13 Images of wounded area in p-HCECs treated with ENA-78. Wound borders were identified (black lines). Wounded area was determined as the area between 2 borders, while wound closure was determined as the difference between the wounded area at 0 hours and 24 hours post injury. Greater wound closure was found in the ENA-78 treatment group compared with the negative control at 24 hours post wounding. Scale bar=50 μm.

FIG. 14 Relative wound closure of p-HCECs treated with ENA-78. A significantly greater relative wound closure compared with the negative control was found in the ENA-78 treatment group 24 hours post injury. Data is presented as mean±SEM. Independent sample T-test was performed. * Significant difference (p-value<0.05).

FIG. 15 Percentage of Ki-67 positive cells at the wounded edge. Percent of Ki-67 positive cells was greater in the ENA-78 treatment group compared with the negative control 24 hours after wounding. Data is presented as mean±SEM. Independent sample T-test was performed. ** Significant difference (p-value<0.01).

FIG. 16 Relative wound closure of corneas treated with ENA-78. A significantly greater wound closure compared with the negative control was found in the ENA-78 treatment group 72 hours after wounding. Data is presented as mean±SEM. Independent sample T-test was performed. ** Significant difference (p-value<0.01).

It is understood that the following description and references to the figures concern exemplary embodiments of the present invention and shall not be limiting to the scope of the claims.

### DETAILED DESCRIPTION

The present invention is based on the surprising finding that CXC chemokines are effective in the treatment and/or prevention of damage or dysfunction of the corneal endothelium (CE). The present inventors also surprisingly found a method of preparing corneal endothelial cells in vitro.

Corneal endothelial cells (CECs) form the CE, which is the innermost layer of the cornea with a thickness of 5 μm. The CE consists of a monolayer of CECs, which is located on the posterior corneal surface. CECs are characterised by a hexagonal shape, which is likely due to apicobasal polarity provided by ZO-1, and the markers ZO-1 as well as the sodium/potassium (Na⁺/K⁺)-ATPase pump. The CE plays several roles in maintaining corneal homeostasis, transparency, and thickness, and CECs regulate aqueous humour flow into and out of the stroma. Transparency depends on the inner integrity of the CE. ZO-1 is an essential sub-membranous protein of the tight junction lining along the apical side of the CECs and plays a major role in the barrier function of the CE. The CE does not only serve as a barrier but also acts as an active ion and solute transporter. CECs allow leakage of solutes and nutrients from the aqueous humour to provide nutrition for stromal keratocytes and corneal epithelial cells. At the same time, CECs pump water via active transport from the stroma and into the aqueous humour to maintain systematic homeostasis, which in turn results in appropriate corneal hydration and transparency. The active transport of fluid out of the stroma depends on the sodium/potassium (Na⁺/K⁺)-ATPase pump, which is a plasma membrane protein pump that mediates the ATP-dependent transport of Na⁺ and K⁺ across the membrane, leading to low internal Na⁺ and high internal K⁺ concentrations. This mechanism regulates corneal homeostasis. Loss of this function could result in corneal oedema, leading to partial or complete blindness.

The CE can be prone to damage or dysfunction. Adult CEC density is approximately 2000-3000 cells/mm². However, CECs do not have regenerative potential in vivo and must be maintained throughout life to provide a functional CE. The number of CECs in the CE declines with age approximately by 0.6%/year. Damage or dysfunction of the CE could lead to partial or total blindness, and the primary treatment is corneal transplantation. Damage to the CECs can be defined as internal and external cellular changes, which can be caused by a variety of stresses (e.g., intraocular surgery or corneal disease). Such cellular damage may be reversible or irreversible depending on the cells' adaptability to the stressor. Dysfunction of the CECs can be defined as an impairment any of the aforementioned functions of CECs, or any combination thereof. CEC dysfunction can be caused by a multitude of external factors including, for instance, ageing or corneal diseases. Because CECs lack proliferative ability, the loss of a significant number of CECs due to irreversible damage or dysfunction can cause significant damage and/or dysfunction of the CE as a whole.

It is noted that CECs generally do not proliferate and this differentiates them from other cell types such as corneal epithelial cells.

Without wishing to be bound by theory, it is considered that the treatment and/or prevention of damage or dysfunction of the CE may be achieved by enhancing proliferation and/or migration of the remaining functional CECs. Enhancing proliferation can be defined as an increase in cell divisions, which effectively causes an increase in the number of cells. Enhancing migration can be defined as increasing the mobility of the cells, for instance, to achieve wound closure.

In some embodiments, the damage or dysfunction of the CE is caused by viral infection, intraocular surgery or Fuchs' endothelial dystrophy (FED), and/or ageing. For example the intraocular surgery is anterior segment surgery. In a further example, the anterior segment surgery is glaucoma surgery or cataract surgery.

According to the present invention, CXC chemokines are used for the treatment and/or prevention of damage or dysfunction of the CE. CXC chemokines are a class of chemokines with two N-terminal cysteines (C) that are separated by one variable amino acid (X). In some embodiments, the CXC chemokine is selected from the group consisting of: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16 and CXCL17. In some embodiments, the CXC chemokine is selected from the group consisting of: CXCL1, CXCL2, CXCL3, CXCL5 or CXCL7. It is understood that CXC chemokines CXCL1, CXCL2, CXCL3, CXCL5 and CXCL7 target cells through the same receptor, the CXCR2 receptor. In some embodiments, the CXC chemokine is CXCL5.

Each of the CXC chemokines may be referred to by alternative names. For example, CXCL1 is also known as GROα, CXCL2 as GROB, CXCL3 as GROγ, CXCL5 as ENA-78, and CXCL7 as NAP-2.

In some embodiments, the CXC chemokine is part of a pharmaceutical composition. In some embodiments, the pharmaceutical composition comprises a pharmaceutically acceptable solvent or carrier system, optionally with an additional ophthalmic excipient. A pharmaceutically acceptable solvent may, for example, be selected from an aqueous solvent such as water, physiological saline and buffer. The pharmaceutically acceptable solvent or carrier system can be defined as the media in which the active is dispersed and may be aqueous or buffer system or likewise. The solvent or carrier system may further contain various additives such as a viscosity agent, a stabilizer, a preservative, a surfactant, an antioxidant, a chelating agent, a pH adjusting agent, a thickener and/or an absorption promoter which are known to a skilled person in art.

The pharmaceutical composition optionally comprises preservative(s) for preventing contamination with microorganisms such as fungi and bacteria. The preservative usable has antibacterial action and antifungal action, and should be non-toxic, non-irritant and applicable to the eye. The pharmaceutical composition may also optionally comprise pH adjusting agents, antioxidants, and/or chelating agents.

In some embodiments, the pharmaceutical composition is formulated as eye drops, intracameral injection, or slow-release insert. An intracameral injection can be defined as the injection of a soluble agent into the anterior chamber. The soluble agent may comprise an active ingredient such as CXCL5. A slow-release insert is an insert that releases the active agent over a prolonged period in comparison to what is known in the art as immediate release. This means that the active agent is available over a longer period of time as it is released in a slow and controlled manner. Slow release may also be referred to as sustained release, delayed release, continuous release, controlled release or retarded release. For example, ocular inserts specifically designed for ophthalmic application can be defined as sterile, thin devices which contain an active agent. Ocular inserts may be of solid or semisolid consistency and are commonly placed into the cul-de-sac or conjuctival sac. They usually contain a polymeric support drug that can be incorporated. They provide sustained release of medication into the eye. The ocular insert may be insoluble, soluble, or bioerodible. A further embodiment of the present invention is a physiological supplement or medicament for ophthalmic use, in the form of eye drops, comprising as active ingredient a CXC chemokine, or one of its pharmaceutically acceptable salts. The eye drops may further contain humidifying agents, and/or antioxidants such as vitamin E, and/or organic and inorganic elements for the regulation of the cellular osmolarity, and/or inorganic elements such as components of enzymes present in the tear film, and/or ophthalmologically acceptable excipients and/or diluents, wherein the physiological supplement or medicament is for use in the treatment and/or prevention of diseases or dysfunction of the CE.

The present invention further provides a method of preparing corneal endothelial cells in vitro, wherein the method comprises:
a. peeling the Descemet membrane together with the endothelial cells from a donor cornea,
b. isolating the primary CECs from the Descemet membrane using one or more cell dissociation reagent(s); and
c. culturing the CECs in medium comprising CXCL5.

In some embodiments, the CECs are arranged as an endothelial cell layer. In some embodiments, the endothelial cell layer is a corneal graft.

In some embodiments, the one or more cell dissociation reagent(s) is/are selected from the group comprising collagenase, trypsin, trypsin-EDTA, elastase and dispase. In some embodiments the cell dissociation reagent is collagenase. In some embodiments the cell dissociation reagent is trypsin.

In some embodiments, the method of the present invention comprises culturing the CECs in medium until they reach confluency, before the cells are cultured in serum free medium comprising CXCL5.

Donor corneas used in the method may be obtained through donations of deceased people and are previously removed from the donor after death. Hence, the method of the invention is an ex vivo method of preparing CECs. Therefore step a. comprises donor cornea ex vivo.

The Descemet membrane can be defined as the membrane that is located between the stroma and the endothelial layer of the cornea. The corneal graft, or cornea transplant, can be defined as replacement corneal tissue that can be used in corneal transplantation.

In certain embodiments, the CXC chemokine may have one or more of the following effects:
increase in CEC migration in vivo;
increase in CEC proliferation in vivo;
treating CE disease and/or dysfunction;
preventing CE disease and/or dysfunction;
enhancing CEC migration and/or proliferation in vitro;
enable generation of corneal grafts; and
enable non-invasive treatment of CE disease and/or dysfunction.
A CXC chemokine for use in the treatment and/or prevention of damage or dysfunction of the corneal endothelium (CE).

The present disclosure may be described by one or more of the following paragraphs:
1. A CXC chemokine for use in the treatment and/or prevention of damage or dysfunction of the corneal endothelium (CE).
2. The CXC chemokine for use according to paragraph 1, wherein the treatment and/or prevention of damage or dysfunction of the CE is achieved by enhancing proliferation and/or migration of the corneal endothelial cells (CECs).
3. The CXC chemokine for use according to paragraph 1 or paragraph 2, wherein the damage or dysfunction of the CE is caused by viral infection, intraocular surgery or Fuchs' endothelial dystrophy (FED), and/or ageing.
4. The CXC chemokine for use according to paragraph 3, wherein the intraocular surgery is anterior segment surgery.
5. The CXC chemokine for use according to paragraph 4, wherein the anterior segment surgery is glaucoma surgery or cataract surgery.
6. The CXC chemokine for use according to any one of the preceding paragraphs, wherein the CXC chemokine is selected from the group consisting of: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16 and CXCL17.
7. The CXC chemokine for use according to any one of paragraphs 1 to 6, wherein the CXC chemokine is selected from the group consisting of: CXCL1, CXCL2, CXCL3, CXCL5 or CXCL7.
8. The CXC chemokine for use according to claim any one of paragraphs 1 to 7, wherein the CXC chemokine is CXCL5.
9. The CXC chemokine for use according to any one of the preceding paragraphs, wherein the CXC chemokine is part of a pharmaceutical composition.
10. The CXC chemokine for use according to paragraph 9, wherein the pharmaceutical composition comprises a pharmaceutically acceptable solvent or carrier system.
11. The CXC chemokine for use according to paragraph 10, wherein the pharmaceutical composition comprises an ophthalmic excipient.
12. The CXC chemokine for use according to any one of paragraphs 9 to 11, wherein the pharmaceutical composition is formulated as eye drops, intracameral injection, or slow-release insert.
13. A method of preparing corneal endothelial cells (CECs) in vitro, wherein the method comprises:
   peeling the Descemet membrane together with the endothelial cells from a donor cornea,
   isolating the primary CECs from the Descemet membrane using one of more cell dissociation reagent(s); and
   culturing the CECs in medium comprising CXCL5.
14. The method of paragraph 13, wherein the CECs are arranged as an endothelial cell layer.
15. The method of paragraph 14, wherein the endothelial cell layer is a corneal graft.
16. The method of any one of claims 13 to 15, wherein the one or more cell dissociation reagent(s) is/are selected from the group comprising collagenase, trypsin, trypsin-EDTA, elastase and dispase.
17. The method of any one of paragraphs 13 to 16, wherein the one or more cell dissociation reagent(s) is/are selected from collagenase or trypsin.
18. A method of treating and/or preventing damage or dysfunction of the CE, comprising administering to a subject in need thereof a therapeutically effective amount of CXC chemokine.
19. The method of paragraph 18, wherein the treatment enhances proliferation and/or migration of CECs.
20. The method of any one of paragraphs 18 or 19, wherein the damage or dysfunction of the CE is caused by viral infection, intraocular surgery or Fuchs' endothelial dystrophy (FED), and/or ageing.
21. The method of paragraph 20, wherein the intraocular surgery is anterior segment surgery.
22. The method of paragraph 21, wherein the anterior segment surgery is glaucoma surgery or cataract surgery.
23. The method of any one of paragraphs 18-22, wherein the CXC chemokine is selected from the group consisting of: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16 and CXCL17.
24. The method of any one of paragraphs 18-23, wherein the CXC chemokine is selected from the group consisting of: CXCL1, CXCL2, CXCL3, CXCL5 or CXCL7.
25. The method of any one of paragraphs 18-24, wherein the CXC chemokine is CXCL5.
26. The method of any one of paragraphs 18-25, wherein the CXC chemokine is part of a pharmaceutical composition.
27. The CXC chemokine for use according to paragraph 26, wherein the pharmaceutical composition comprises one or more pharmaceutically acceptable excipient(s).
28. The CXC chemokine for use according to paragraph 27, wherein the pharmaceutical excipient(s) is selected from carriers, excipients and/or diluents.
29. The method of any one of paragraphs 26 to 28, wherein the pharmaceutical composition is formulated as eye drops, intracameral injection, or slow-release insert.
30. Use of a CXC chemokine in the manufacture of a medicament for the treatment and/or prevention of damage or dysfunction of the corneal endothelium (CE).
31. The use according to paragraph 30, wherein the treatment and/or prevention of damage or dysfunction of the CE is achieved by enhancing proliferation and/or migration of the corneal endothelial cells (CECs).
32. The use according to paragraph 30 or paragraph 31, wherein the damage or dysfunction of the CE is caused by viral infection, intraocular surgery or Fuchs' endothelial dystrophy (FED), and/or ageing.
33. The use according to paragraph 32, wherein the intraocular surgery is anterior segment surgery.
34. The use according to paragraph 33, wherein the anterior segment surgery is glaucoma surgery or cataract surgery.
35. The use according to any one of paragraphs 30 to 34, wherein the CXC chemokine is selected from the group consisting of: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16 and CXCL17.
36. The use according to any one of paragraphs 30 to 35, wherein the CXC chemokine is selected from the group consisting of: CXCL1, CXCL2, CXCL3, CXCL5 or CXCL7.
37. The use according to any one of paragraphs 30 to 36, wherein the CXC chemokine is CXCL5.
38. The use according to any one of paragraphs 30 to 37, wherein the CXC chemokine is part of a pharmaceutical composition.
39. The use according to paragraph 38, wherein the pharmaceutical composition comprises a pharmaceutically acceptable solvent or carrier system.
40. The use according to paragraph 39, wherein the pharmaceutical composition comprises an ophthalmic excipient.
41. The use according to any one of paragraphs 38 to 40, wherein the pharmaceutical composition is formulated as eye drops, intracameral injection, or slow-release insert.
42. A pharmaceutical composition comprising a CXC chemokine to treat and/or prevent damage or dysfunction of the corneal endothelium (CE).
43. The pharmaceutical composition according to paragraph 42, wherein the treatment and/or prevention of damage or dysfunction of the CE is achieved by enhancing proliferation and/or migration of the corneal endothelial cells (CECs).
44. The pharmaceutical composition according to paragraph 42 or 43, wherein the damage or dysfunction of the CE is caused by viral infection, intraocular surgery or Fuchs' endothelial dystrophy (FED), and/or ageing.
45. The pharmaceutical composition according to paragraph 44, wherein the intraocular surgery is anterior segment surgery.
46. The pharmaceutical composition according to paragraph 45, wherein the anterior segment surgery is glaucoma surgery or cataract surgery.
47. The pharmaceutical composition according to any one of paragraphs 42 to 46, wherein the CXC chemokine is selected from the group consisting of: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16 and CXCL17.
48. The pharmaceutical composition according to any one of paragraphs 42 to 47, wherein the CXC chemokine is selected from the group consisting of: CXCL1, CXCL2, CXCL3, CXCL5 or CXCL7.
49. The pharmaceutical composition according to any one of paragraphs 42 to 48, wherein the CXC chemokine is CXCL5.
50. The pharmaceutical composition according to any of paragraphs 42 to 49, wherein the pharmaceutical composition comprises a pharmaceutically acceptable solvent or carrier system.
51. The pharmaceutical composition according to paragraph 50, wherein the pharmaceutical composition comprises an ophthalmic excipient.
52. The pharmaceutical composition according to paragraphs 50 or 51, wherein the pharmaceutical composition is formulated as eye drops, intracameral injection, or slow-release insert.

### Examples

### Materials and Methods

### Cell Proliferation Study

MTT was used as an indirect method to measure cell proliferation. HCECLs (Human Corneal Endothelial Cell line, B4G12; DSMZ, Germany) were seeded at a density of 250 cells/mm² in 96 well plates. Cell culture medium was changed to medium comprising 1, 5, 10 or 20 ng/ml of ENA-78 (R&D system, cat no. 254-XB-025/CF) the following day. HCECLs treated with medium comprising 10 ng/ml of FGFb (Gibco®, cat no. PHG0026) served as positive control in this study, because FGFb has previously been found to be a promoting factor of CEC proliferation and migration.

Cell proliferation was monitored at day 1 and 3 using MTT. A volume of 10 μL of MTT (Sigma-Aldrich) and 100 μL of cell culture medium were added into each well of the cell culture plates. The cell culture plates were incubated for 3 hours at 37° C. Next, 100 μL of solubilization solution were added into each well, protected from light, for overnight incubation at room temperature. The absorbance was then measured with a microplate reader (Tecan Saphire) at 570 nm. All absorbance measurements were corrected by the absorbance of the medium without cells. Three technical replications were performed.

Cell counting was applied in this study to monitor cell proliferation. HCECLs were seeded at a density of 250 cells/mm² in 24 well plates. Cell culture medium was changed to medium comprising 1, 5, 10 or 20 ng/mL of ENA-78 the following day. Cell numbers were counted on day 1 and day 3. HCECLs were dissociated from the cell culture plates using trypLE™ Express. The number of cells was determined using a Neubauer hemocytometer (Neubauer-improved counting chamber, Marienfeld, Germany). Each independent experiment was performed three times.

### Wound Healing Assay

HCECLs were seeded at a density of 250 cells/mm² in 6 well plates and cultured until the cells reached confluence. HCECLs were then starved by feeding with medium without serum for 24 hours. The following day, the cell culture medium was removed, and the cells were scratched with a 200 μl pipette tip. 1 mL of DPBS was added to wash away any excess cell debris. Media comprising either 20 ng/ml of ENA-78 or 10 ng/mL of FGFb were added at a volume of 2 ml/well. Wound closure in either condition was assessed using light microscopy (Eclipse TS100, Nikon) at 10× magnification on day 0, 1, 2 and 3. Percent of wound closure was measured using ImageJ and calculated according to the following equation. The results were compared with the control groups (cells left untreated after wound assay). The experiment was performed three times.

### Equation 1-Percent of Wound Closure

Aₜ₌ₒₕ₌ₐᵣₑₐ of the wound measured immediately after scratching (t=0 hour),
A_{t=Δh=area} of the wound measured h hours after the scratching

$$Wound Closure \% = [ \frac{{A}_{t = 0 h} - {A}_{t = Δ h}}{{A}_{t = 0 h}} ] \times 100 \%$$

### Identify ENA-78/CXCR2 Pathway

CXCR2 is a specific receptor of ENA-78. It is known that ENA-78 selectively binds to CXCR2, a G-protein coupled receptor. It was hypothesised that the promoting effect of ENA-78 on CEC proliferation and wound healing was mediated by CXCR2 (ENA-78/CXCR2). Any interference or factors disturbing this pathway should result in a decrease in HCECL proliferation. To examine the impact of CXCR2 on CEC proliferation, HCECLs were treated with a CXCR2 antibody (R&D system, cat no. MAB331-100) and HCECL proliferation was assessed HCECLs were seeded at a density of 250 cells/mm² in 96 well plates. The following day, the cell culture medium was changed to medium comprising 20 ng/ml of ENA-78 (ENA-78), 2 μg/mL of CXCR2 antibody (CXCR2), or 20 ng/ml of ENA-78 and 2 μg/mL of CXCR2 antibody (ENA-78+CXCR2). Cell proliferation was assessed on day 3 using an MTT assay.

Wound healing was examined using a wound healing assay as described above. Briefly, HCECLs were starved for 24 hours, the cell culture medium was subsequently removed, and the cells were scratched with a 200 μl pipette tip. Media comprising either 20 ng/ml of ENA-78 (ENA-78), 2 μg/mL of CXCR2 antibody (CXCR2), or 20 ng/ml of ENA-78 with 2 μg/mL of CXCR2 antibody (ENA-78+CXCR2) were added at a volume of 2 ml/well. The wounded areas were monitored every day for 3 days. The percent of wound closure was calculated as described above. The results were compared with the control groups (cells left untreated after wound assay). The experiment was performed three times.

### Identify ENA-78/ERK Pathway

CXCR2 is a G-protein coupled receptor that can activate MAPK/ERK pathway which is strongly link to cellular proliferation. The p-ERK1/2 is the activated form of ERK, a key kinase that can activate several transcription factors related to cell proliferation and migration. It was hypothesised that the promoting effect of ENA-78 on CEC proliferation and wound healing was sent through ENA-78/ERK pathway which activated p-ERK1/2 protein expression. The expression of p-ERK/1/2 protein in HCECLs was evaluated by western blot analysis. HCECLs were cultured in 6 cm cell culture dishes at a density of 250 cells/mm². Upon reaching 60% confluency, the cells were starved overnight in medium without serum. Cell lysate for the control group was collected the following day. The medium was then changed to medium comprising 20 ng/ml ENA-78, and cells were incubated for 30, 60 or 120 minutes. Cell lysates were collected, and p-ERK1/2 expression was identified using a p-ERK/1/2 antibody (Cell signaling, cat no. 9101S). The immunoblot was scanned using (chemiDoc™ XRS+, Bio-Rad) and quantified using ImageLab 5.0 Software. Band intensity was measured using ImageJ. The results of every band were compared with the results for the GAPDH control.

### Cell Characterization

HCECLs were seeded in 8-well permanox chamber slides (Nunc®, Lab-Tek®) at a density of 2×10⁴ cells/well and cultured in medium comprising 20 ng/ml of ENA-78. Upon reaching confluency, the medium was removed, and the cells were washed with DPBS. For cell fixation, 4% paraformaldehyde was added for 20 minutes at room temperature. The slides were washed three times with DPBS for 20 minutes and blocked with 5% (V/V) goat serum and permeabilised with 0.5% (V/V) Triton-X 100 for 1 hour at room temperature. After blocking, the slides were washed three times with DPBS for 20 minutes. The slides were incubated overnight with the primary antibodies (1:500), ZO-1 (BD scientific, cat no. 610966) and Na⁺/K⁺-ATPase (Santa-Cruz, cat no. SC-71638), at 4° C. The slides were washed three times with DPBS and incubated with Alexa Fluor 488 secondary antibody (1:500) in DPBS protected from light, for 1 hour at room temperature. The slides were washed three times with DPBS and mounted using VECTASHIELD mounting medium with DAPI. The slides were visualized using confocal microscopy (LSM 700, Zeiss).

p-HCEC wound healing, an in vitro study

CECs were isolated from three different human donor corneas (p-HCEC). The average donor age was 67.5 years. The cells were seeded at a density of 1,500 cells/mm² in 8-well permanox chamber slides and cultured until they reached confluency. The cells were starved by feeding with medium without serum for 24 hours. The following day, the cell culture medium was removed, and the cells were scratched with a 200 μl pipette tip. 1 mL of DPBS was added to wash away the excess cell debris. Medium comprising 20 ng/ml of ENA-78 was subsequently added. Wounded closure was assessed using light microscopy (Eclipse TS100, Nikon) at 10× magnification on day 1. Percent of wound closure was measured using ImageJ. The results were compared with the control groups (untreated cells).

At the end of the study, proliferation of p-HCECs was also assessed. Ki-67 was used as a marker of proliferation to determine the proliferation capability of p-HCECs after wounding. The cells were fixed and blocked as described above. Ki-67 antibody (1:500), Alexa Fluor 488 secondary antibody (1:500) and VECTASHIELD mounting medium with DAPI were used. The slides were visualized using confocal microscopy (LSM 700, Zeiss). The percentage of positive Ki-67 cells was calculated as indicator of proliferation.

### P-HCEC Wound Healing, an Ex Vivo Study

Full-thickness donor corneas were cut into four pieces each. Wounds were created by removing p-HCECs using a silicone tube. The wounded cornea was stained with trypan blue to evaluate the area of wounding, as the denuded area was stained blue. Trypan blue is routinely used as a vital stain to access viability of p-HCECs. The staining is based on the concept that trypan blue cannot permeate the cell membrane of viable p-HCECs. Trypan blue permeates the damaged cell membrane of severely damaged and dead cells and stains the cell nucleus, as well as the area of Descemet membrane denuded of p-HCECs. The cornea was incubated in medium comprising 20 ng/ml of ENA-78 for 3 days, and the wounded area was monitored every single day using a stereomicroscope (SMZ 1500, Nikon). Percent of wound closure was compared between the ENA-78 and the control groups (untreated cells).

At the end of the study, 0.2% alizarin red was applied to evaluate p-HCEC morphology. Alizarin red is a water-soluble sodium salt of Alizarin sulfonic acid. It binds to the calcium at the cell tight junction to form a lake pigment which is presented in red colour. It is commonly used for endothelial cell density evaluation and cell morphology visualization as it stains the intercellular border of p-HCECs. A stock solution of alizarin red was prepared by mixing 0.2 g of alizarin red powder with 100 mL of DPBS. The solution was stirred for 3 hours and subsequently filtered with Whatman filter paper No. 1 to remove undissolved sediment. The corneas were washed briefly in DPBS and alizarin red was added dropwise to cover the endothelium. After 3 minutes, the stain was poured off and the corneas were washed twice in DPBS. Cell morphology was observed using a stereomicroscope (SMZ 1500, Nikon).

### Example Method for Preparing CECs In Vitro

Donor corneas are obtained and washed with a sterile DPBS (Gibco®). The cornea is then placed on a corneal trephine platform (Coronet®) and the endothelial surface is stained with tryptan blue solution (0.4%) (Gibco®) to determine the viability of the cells. Subsequently, the cornea is washed twice in DPBS. Then, the Descemet membrane is peeled together with the endothelial cells using jeweler forceps (Altomed). The Descemet membrane and endothelial cells are treated with 2 mg/mL collagenase type 1 (Gibco®) in Human Endothelial Serum-Free Medium (Gibco®) for 2 hours at 37° C. Next, the resulting solution is centrifuged at 1,000 rpm for 5 minutes, and the resulting cell pellet is resuspended in 1 mL trypLE™ Express (Gibco®), followed by centrifuging at 1,000 rpm for 5 minutes to dissociate the cells into single cells. Cells are then counted and plated at a cell seeding density of 1,500 cell/mm². The CECs are cultured in medium consisting of 1:1 (V/V) Ham's F-12 Nutrient Mixture and Medium 199 (Gibco®), 5% (V/V) fetal bovine serum (FBS) (Gibco®), 0.5% (V/V) insulin-transferrin-selenium (Gibco®), 0.1% (V/V) L-ascorbic acid 2 phosphate (Sigma-Aldrich), 10 ng/mLRecombinant human FGF (Gibco®), 10 μM Rho-associated kinase (ROCK) Y27632 inhibitor and 1% (V/V) Penicillin-Streptomycin solution (Gibco®) until the CECs reach confluency. Finally, the medium is changed to medium without serum comprising CXCL5 (the medium consisting of 1:1 (V/V) Ham's F-12 Nutrient Mixture and Medium 199 (Gibco®), 2.5% (V/V) Albumax (Gibco®), 0.5% (V/V) insulin-transferrin-selenium (Gibco®), 0.1% (V/V) L-ascorbic acid 2 phosphate (Sigma-Aldrich), 20 ng/ml of CXCL5 and 1% (V/V) Penicillin-Streptomycin solution (Gibco®)).

### Results

### Effect of ENA-78 on CEC Proliferation

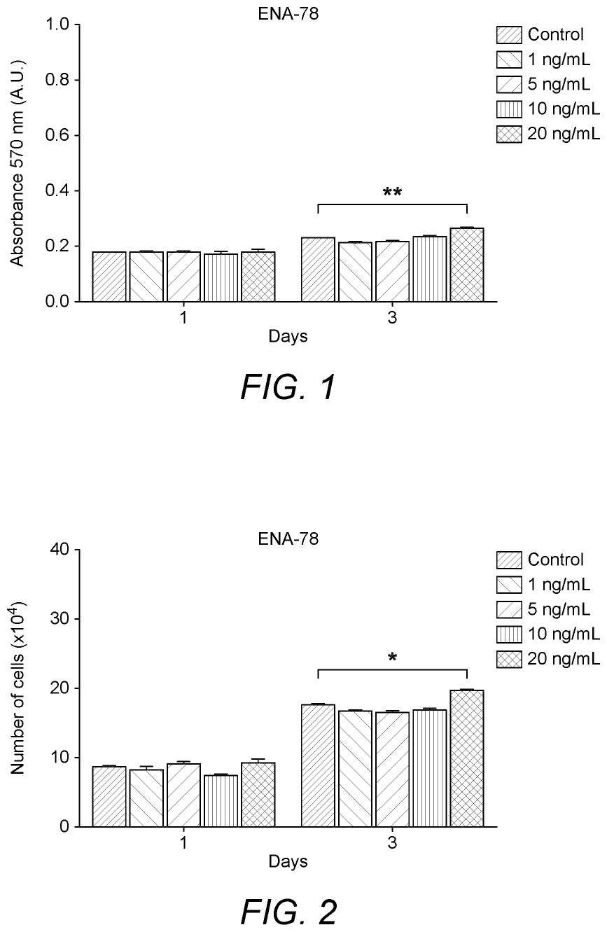

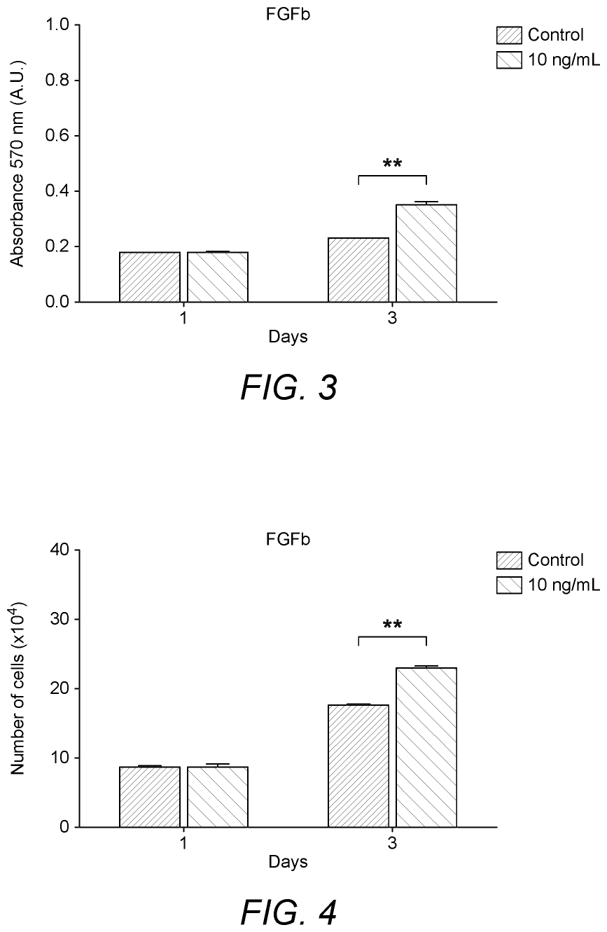

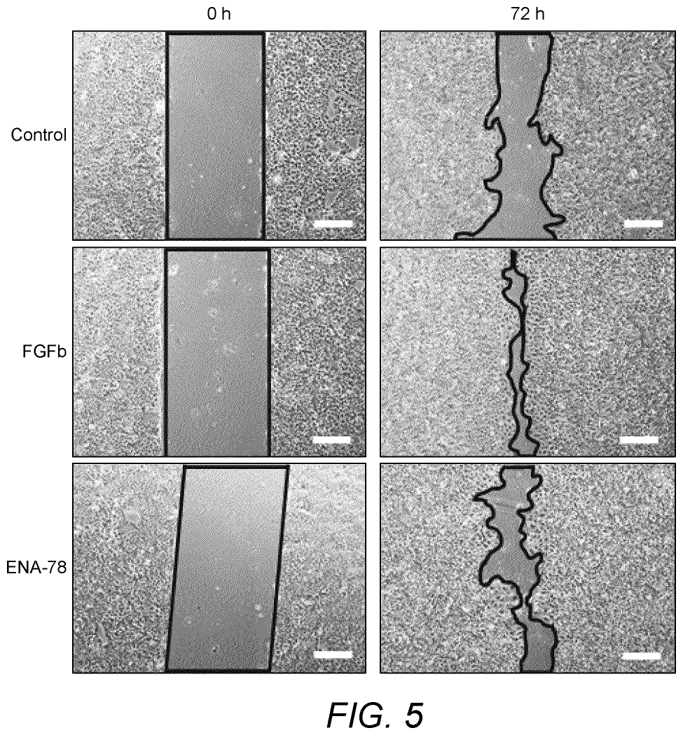

ENA-78 was found to promote CEC proliferation and wound healing in in vitro studies using HCECLs. A statistically significant increase in CEC proliferation and cell number (p-value<0.01 and <0.05) was found after culturing HCECLs in medium comprising 20 ng/mL of ENA-78 for 3 days followed by MTT and cell counting, respectively (FIGS. 1 and 2, respectively). FGFb has previously been found to promote the proliferation and migration of CECs. Therefore, cells treated with 10 ng/ml of FGFb following the same protocol as for treatment with ENA-78 served as a positive control (FIGS. 3 and 4). These results from the cell proliferation study show that ENA-78 promotes CEC proliferation.

### Effect of ENA-78 on CEC Wound Healing

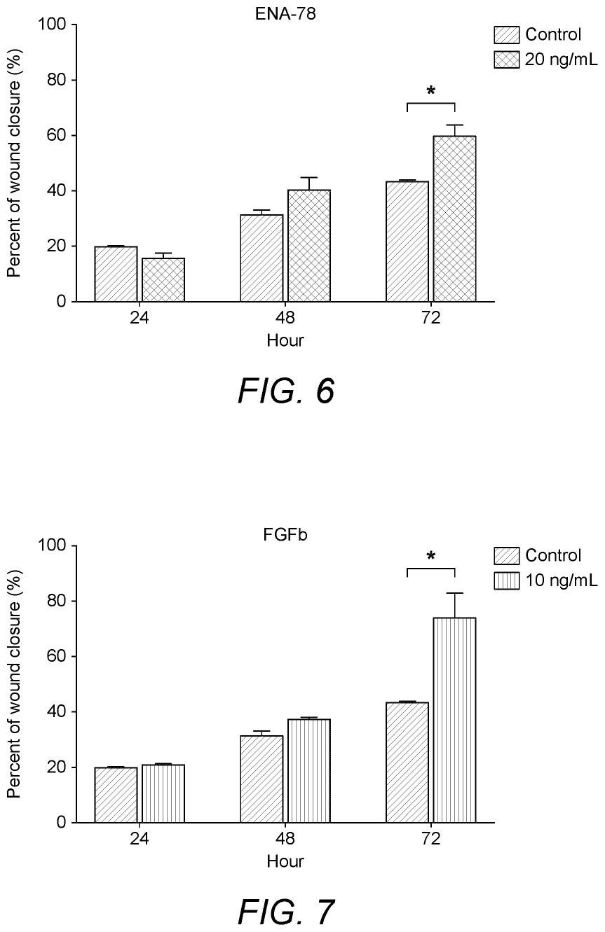

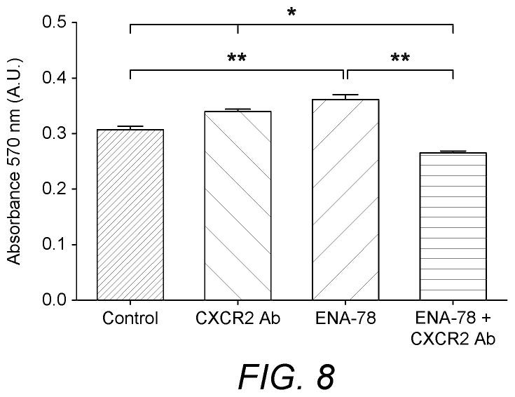

CEC wound healing was observed using an in vitro wound healing assay (FIG. 5). The percent of wound closure increased over time for those wounds treated with ENA-78, as well as the untreated negative control wounds and the positive control wounds treated with FGFb (FIGS. 6 and 7). Wound closure increased from about 20% to about 40% in the ENA-78 treatment group and from about 20% to about 30% in the negative control group 24 to 48 hours post injury, respectively (FIG. 6). From 48 to 72 hours post injury, wound closure increased continuously from about 40% to about 60% in the ENA-78 treatment group and from 30% to 45% in the negative control group (FIG. 6). At 72 hours post injury, the percent of wound closure in the ENA-78 treatment group was significantly greater than the percent of wound closure in the negative control group (p-value<0.05) (FIG. 6). Equally, the positive control showed that the percent of wound closure was significantly greater in the FGFb treatment group than in the negative control group at 72 hours post injury (p-value<0.05) (FIG. 7). In summary, these results showed that ENA-78 promotes CEC wound healing.

### ENA-78 Signalling Pathway

To examine the ENA-78/CXCR2 pathway, HCECLs were treated with a CXCR2 antibody (CXCR2 Ab) after wounding. Cell proliferation was examined using MTT assay at day 3 post injury as detailed above. HCECL cultured in medium comprising ENA-78 showed the highest cell proliferation (FIG. 8). By contrast, HCECLs treated with ENA-78+CXCR2 Ab showed the lowest cell proliferation (FIG. 8). This difference between HCECLs in the ENA-78 treatment group as compared with the negative control group (untreated) and ENA-78+CXCR2 Ab treatment group was statistically significant (p-value<0.01). These results suggest that ENA-78 mediates CEC proliferation via CXCR2.

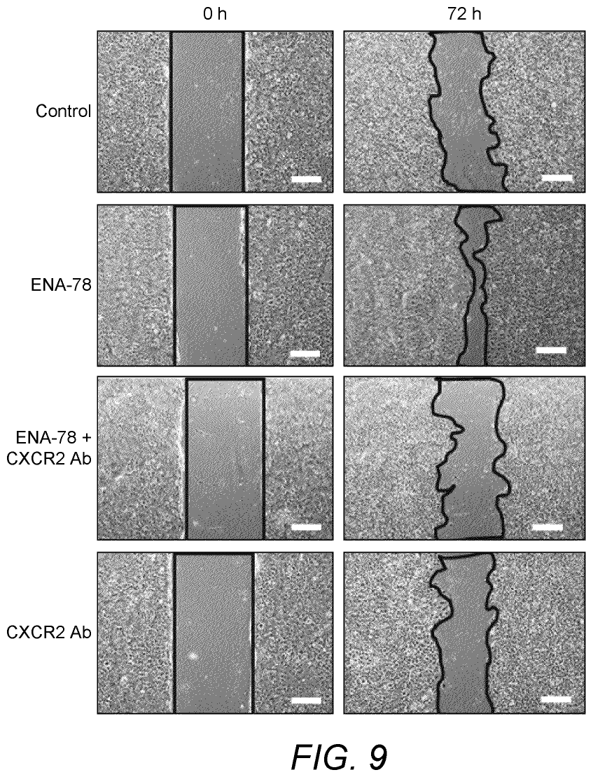

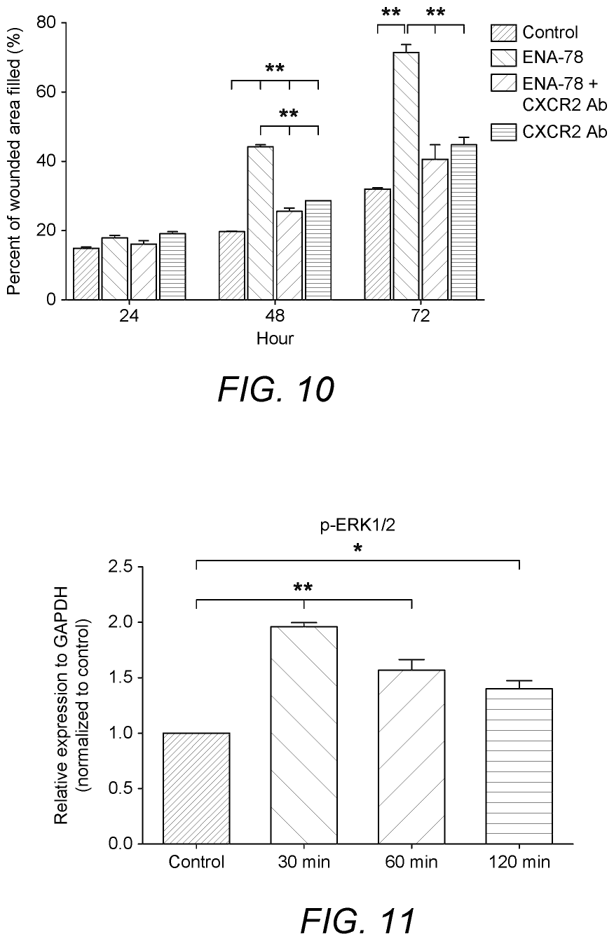

Besides the cell proliferation assay, the percentage of wound closure in the different treatment groups was also assessed. The percent of wound closure increased over time in every group (FIG. 9). Percent of wound closure statistically significantly increased in the ENA-78, ENA-78+CXCR2 Ab and CXCR2 Ab treatment groups as compared to the negative control group 48 hours post injury (p-value<0.01) (FIG. 10). 72 hours post injury, percent of wound closure significantly increased in the ENA-78 treatment group as compared with the negative control group (p-value<0.01) (FIG. 10). Notably, the percent of wound closure in the ENA-78 treatment group was significantly greater than that of the ENA-78+CXCR2 Ab treatment group at 48 and 72 hours post injury (p-value<0.01) (FIG. 10). The greatest percentage of wound closure was determined in the ENA-78 treatment group, followed by the CXCR2 Ab and ENA-78+CXCR2 Ab treatment groups, respectively (FIG. 10). These results suggest that ENA-78 mediates CEC wound healing via CXCR2.

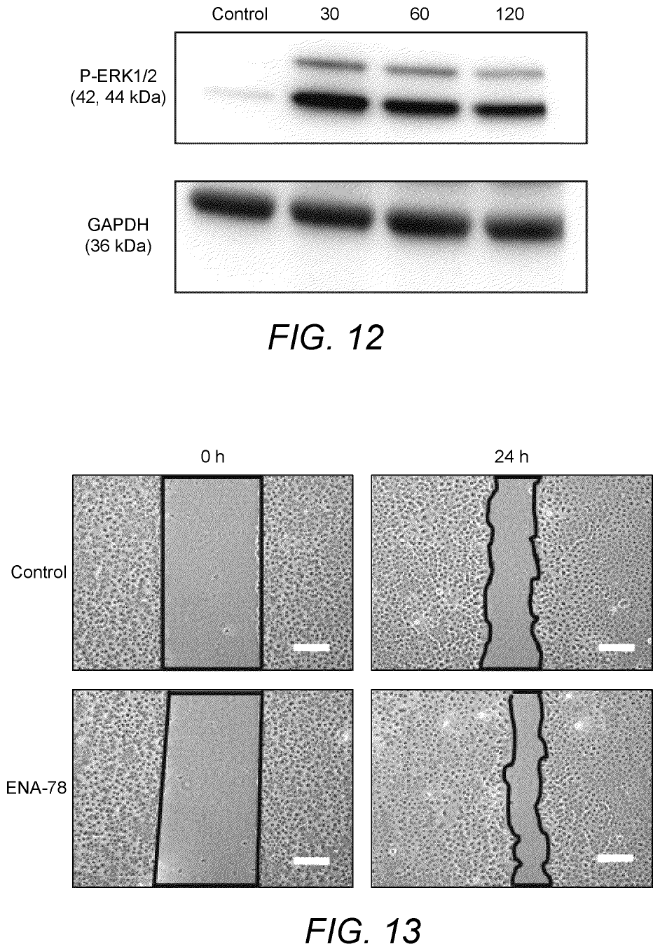

Expression levels of the p-ERK/1/2 protein were evaluated using Western blot. HCECLs were incubated in medium comprising 20 ng/mL ENA-78 for 30, 60 or 120 minutes, and the expression of p-ERK1/2 protein was detected by Western blot analysis. The expression of p-ERK1/2 proteins was found as early as 30 minutes after the cells were exposed to ENA-78, with the greatest expression levels being determined at 30 minutes post treatment (FIGS. 11 and 12).

The results from the cell signalling pathway studies showed that ENA-78 acts via binding to CXCR2 and the signal was mediated by p-ERK1/2.

Characteristics of CEC after Exposed to ENA-78

Previous studies have used Na⁺/K⁺-ATPase and zonula occludens-1 (ZO-1) as typical markers of functional monolayer CECs. The presence of ZO-1 at the cell membrane shows the hexagonal shapes of CECs, and the Na⁺/K⁺-ATPase marker is widely used to identify CEC function. Hence, immunocytochemistry was performed to detect expression of the markers by HCECLs after treatment with ENA-78 to confirm that ENA-78 treated CECs remain functional monolayer CECs. The expression of Na⁺/K⁺-ATPase and ZO-1 were similar between ENA-78 treatment and the negative control groups. Briefly, ENA-78 treated CECs retained CEC characteristics by expressing ZO-1 and Na⁺/K⁺-ATPase cell surface markers.

Effect of ENA-78 on p-HCEC Wound Healing and Proliferation, an In Vitro Study

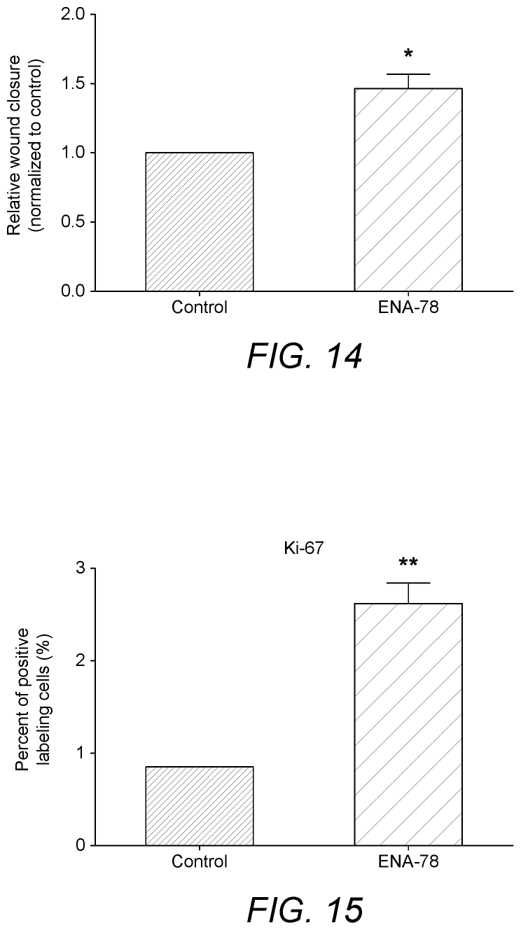

CECs used in the following in vitro study were isolated and obtained from human donor corneas and are referred to as primary human corneal endothelial cells (p-HCECs). The cells were cultured until reaching confluency. Subsequently, the wound healing assay was performed as described previously (p-HCEC wound healing, an in vitro study) to evaluate wound healing capability. At 24 hours post injury, wound closure was significantly greater in the group of p-HCEC cultured in medium comprising ENA-78 compared with the negative control (p-value<0.05) (FIGS. 13 and 14).

Cell proliferation of p-HCECs was also observed after an in vitro wound healing assay was conducted as described in the p-HCEC wound healing, an in vitro study. The commonly known marker of proliferation Ki-67 was used to identify proliferation capability of p-HCEC after wounding. p-HCECs were stained for Ki-67 by immunocytochemistry at day 24 hours post injury. Ki-67 positive cells were found at the wounded edge. Notably, the percentage of Ki-67 positive cells was significantly greater in the ENA-78 treatment group compared with the negative control groups (2.5% vs. <1%, respectively, p-value<0.05) (FIG. 15). These results suggest that ENA-78 promotes p-HCEC wound healing and proliferation.

Effect of ENA-78 on p-HCEC Migration in Wound Healing Process, an Ex Vivo Study

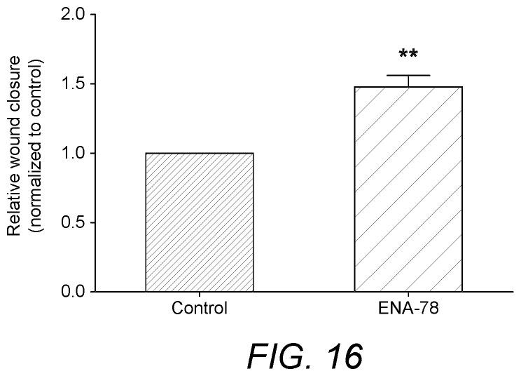

Full-thickness donor cornea was cut to create a wound. The area of wounding was evaluated using trypan blue staining. The corneas were then incubated in medium comprising 20 ng/mL of ENA-78 for 72 hours and the wounded area was monitored at 24, 48 and 72 hours post injury. Wound closure areas were compared between the ENA-78 treatment and the negative control groups. At 72 hours post injury, narrowing of the wounded area was found in both groups. However, wound closure was found to be greater in ENA-78 treatment group as compared with the negative control (FIG. 16). Notably, complete wound closure was observed in the ENA-78 treatment group. Relative wound closure was significantly greater in the ENA-78 treatment group compared with the negative control group (p-value<0.01) (FIG. 16).

Finally, the corneas were stained with alizarin red to evaluate p-HCEC morphology. Consistent small hexagonal cell shapes were found in the areas without wounding. Meanwhile, irregular large-elongated cell shapes were observed at the wounded edges of both the ENA-78 treatment and negative control groups. This cell shape is known to indicate morphology of p-HCECs during migration. Meanwhile, polygonal shape cells with small cell bodies were present at the area of complete wound closure in the ENA-78 treatment group.

In summary, the results of the ex vivo study confirm the effect of ENA-78 on cell migration in the process of CEC wound healing. This study highlights the possibility of using ENA-78 as a medical treatment alternative to corneal transplantation.

### CONCLUSION

The effect of ENA-78 on CEC proliferation and wound healing was described for the first time in this study. It was found that ENA-78 promotes CEC proliferation and wound healing via binding to CXCR2, G-couple protein receptor, and activated ERK phosphorylation (p-ERK) signalling pathway. CECs retained their specific characteristics and expressed CEC markers (ZO-1 and Na⁺/K⁺-ATPase) after treatment with ENA-78. The effect of ENA-78 on CEC migration was also observed in an ex vivo study using human donor corneas. Wound closure was greater in the group of the corneas incubated in medium comprising ENA-78. The results of the alizarin red staining suggest that this wound closure was modulated by the process of cell migration, resulting in a marked elongation of CECs perpendicular to the wound.

1.-10. (canceled)

11. A method of treating and/or preventing damage or dysfunction of the CE, comprising administering to a subject in need thereof a therapeutically effective amount of CXC chemokine.

12. The method of claim 11, wherein the treatment enhances proliferation and/or migration of CECs.

13. The method of claim 11, wherein the damage or dysfunction of the CE is caused by viral infection, intraocular surgery or Fuchs' endothelial dystrophy (FED), and/or ageing.

14. The method of claim 13, wherein the intraocular surgery is anterior segment surgery.

15. The method of claim 14, wherein the anterior segment surgery is glaucoma surgery or cataract surgery.

16. The method of claim 11, wherein the CXC chemokine is selected from the group consisting of: CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16 and CXCL17.

17. The method of claim 11, wherein the CXC chemokine is selected from the group consisting of: CXCL1, CXCL2, CXCL3, CXCL5 or CXCL7.

18. The method of claim 11, wherein the CXC chemokine is CXCL5.

19. The method of claim 11, wherein the CXC chemokine is part of a pharmaceutical composition.

20. The method of claim 19, wherein the pharmaceutical composition comprises one or more pharmaceutically acceptable excipient(s).

21. The method of claim 20, wherein the pharmaceutical excipient(s) is selected from carriers, excipients and/or diluents.

22. The method of claim 19, wherein the pharmaceutical composition is formulated as eye drops, intracameral injection, or slow-release insert.

23. A method of preparing corneal endothelial cells (CECs) in vitro, wherein the method comprises:
a. peeling a Descemet membrane together with the endothelial cells from a donor cornea,
b. isolating the primary CECs from the Descemet membrane using one of more cell dissociation reagent(s); and
c. culturing the CECs in a medium comprising CXCL5.

24. The method of claim 23, wherein the CECs are arranged as an endothelial cell layer.

25. The method of claim 24, wherein the endothelial cell layer is a corneal graft.

26. The method of claim 23, wherein the one or more cell dissociation reagent(s) is/are selected from the group comprising collagenase, trypsin, trypsin-EDTA, elastase and dispase.

27. The method of claim 23, wherein the one or more cell dissociation reagent(s) is/are selected from collagenase or trypsin.
